(19) United States
(12) Patent Application Publication
(10) Pub. No.: US 2024/0125756 A1
(43) Pub. Date: Apr. 18, 2024

(54) PRECISION FARMING SYSTEM WITH SCALED SOIL CHARACTERISTICS

(71) Applicant: Trace Genomics, Inc., Redwood City, CA (US)

(72) Inventors: Ronald O. Zink, Mercer Island, WA (US); Maria Mooshammer, El Cerrito, CA (US); Poornima Parameswaran, Menlo Park, CA (US); Sheng-Yang Matthew Goh, San Franciisco, CA (US); Tyler Barnum, Redwood City, CA (US)

(21) Appl. No.: 18/046,123

(22) Filed: Oct. 12, 2022

(51) Int. Cl. G01N 33/24; A01G 7/00; C12Q 1/6888

(52) U.S. Cl. CPC G01N 33/24; A01G 7/00; C12Q 1/6888; G01N 2033/245

(57) ABSTRACT

A method of selecting a treatment recommendation for nitrogen loss in a field includes measuring a first characteristic of a soil sample that is related to biologic nitrogen loss to produce a measured value. A second characteristic of the soil sample is measured and is used to select a sample group for the soil sample. The measured value is scaled based on the soil sample group the soil sample is placed into to form a scaled measure and the scaled measure is used to select a treatment recommendation.

### BACKGROUND

Precision farming systems divide fields into zones, determine the current conditions in each zone and report those conditions to farmers so that the farmer can decide what actions to take in each zone. Example conditions include nutrient levels, such as nitrogen and phosphorus levels, organic matter levels and moisture levels.

The discussion above is merely provided for general background information and is not intended to be used as an aid in determining the scope of the claimed subject matter. The claimed subject matter is not limited to implementations that solve any or all disadvantages noted in the background.

### SUMMARY

A method of selecting a treatment recommendation for nitrogen loss in a field includes measuring a first characteristic of a soil sample that is related to biologic nitrogen loss to produce a measured value. A second characteristic of the soil sample is measured and is used to select a sample group for the soil sample. The measured value is scaled based on the soil sample group the soil sample is placed into to form a scaled measure and the scaled measure is used to select a treatment recommendation.

In accordance with a further embodiment, a method includes using a soil texture of a soil sample to select a group of soil samples. A characteristic of the soil sample is measured to produce a measured value that is scaled based on measured values determined for soil samples in the group of soil samples to form a scaled value. The scaled value is displayed instead of the measured value so as to improve a precision farming system.

In accordance with a still further embodiment, a method includes using a soil structure of a soil sample to select a sample group for the soil sample. A denitrification count of the soil sample is determined and then is scaled relative to denitrification counts of other soil samples in the sample group to form a scaled denitrification count. A user interface is then displayed depicting the scaled denitrification count.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is a block diagram of a system for generating nitrogen leaching risks and scaled biologic N loss risk for a soil sample.

FIG. 2 is a flow diagram of a method of processing a soil sample using the system of FIG. 1.

FIG. 3 is a flow diagram of a method of performing biologic content tests.

FIG. 4 is a flow diagram of a method setting treatment recommendations for a zone of a field.

FIG. 5 is a block diagram showing elements used in the method of FIG. 4.

FIG. 6 is a graph showing treatment regions for combinations of leaching risk and unscaled biologic N loss risk.

FIG. 7 is the graph of FIG. 6 overlayed with treatment boxes based on scaled biologic N loss risk.

FIG. 8 is an example user interface depicting scaled biologic N loss risk for zones of a field on a map.

FIG. 9 is an example user interface depicting scaled oxygen availability values for zones of a field on a map.

FIG. 10 is an example user interface depicting scaled denitrification counts for zones of a field on a map.

FIG. 11 is an example user interface depicting nitrogen leaching risk for zones of a field on a map.

FIG. 12 is an example of a user interface depicting scaled biologic N loss risks for zones across a collection of fields.

FIG. 13 is an example of a user interface depicting scaled oxygen availability values for zones across a collection of fields.

FIG. 14 is an example of a user interface depicting scaled denitrification counts across a collection of fields.

FIG. 15 is an example of a user interface depicting nitrogen leaching risk across a collection of fields.

FIG. 16 provides a block diagram of a computing device used to implement the various embodiments.

### DETAILED DESCRIPTION

Prior art precision farming systems are difficult for farmers to use because the user interfaces return confusing nutrient information that does not by itself indicate whether the farmer should take action to try to improve yields. Specifically, under the prior art, it is possible for two fields, one needing treatment and one not needing treatment, to generate the same nutrient value. It is not that prior art precision farming systems are incorrectly measuring the nutrient value in the field. Instead, the present inventors have discovered that prior art precision farming systems erroneously assume that every zone in every field can achieve the maximum nutrient value possible in any field anywhere. The present inventors have discovered that this is not true and have further discovered certain co-factors that allow samples to be grouped together to define attainable nutrient value ranges for a field. By using these ranges of attainable values, the embodiments described below improve precision farming systems by providing nutrient information that is less confusing to farmers. Specifically, in the embodiments below, the nutrient values returned to the farmer are directly indicative of the degree to which the yield in the zone can be improved since the nutrient values are scaled relative to nutrient values of other zones in the sample group. This allows the farmer to quickly assess whether they should take action to improve some aspect of the field.

The co-factors that have been identified are characteristics of the soil samples that allow the soil samples to be grouped. In particular, the co-factors form groups of soil samples that each have smaller ranges of values for the nutrient values of interest than the range provided by all of the soil samples. In accordance with one embodiment, some of the smaller ranges overlap parts of the smaller ranges of other sample groups. Others of the smaller ranges do not overlap any other sample group's range of nutrient values.

One example of nutrient values produced by the present embodiments are values related to nitrogen loss risk in a zone. Specifically, nutrient values representing: leaching risk, denitrification risk and oxygen availability. Leaching risk is a measure of degree to which the soil is prone to losing nitrate through water transport. Denitrification risk is a measure of the degree to which microbes in the soil will convert nitrate to N₂O and/or N₂ gases that will then leave the soil. Oxygen availability is a measure of soil conditions that are favorable for denitrification.

The present inventors have discovered that co-factors, such as soil texture, can be used to group soil samples so as to define ranges of possible values of denitrification risk and oxygen availability for each sample. Thus, within a group of samples, the present embodiments are able to set a respective maximum value and a respective minimum value for each of the denitrification risk and oxygen availability. Because these ranges better reflect what is attainable for the zone that a sample was taken from, the resulting values returned to the farmer more clearly indicate whether the yield of the zone could be improved with treatment.

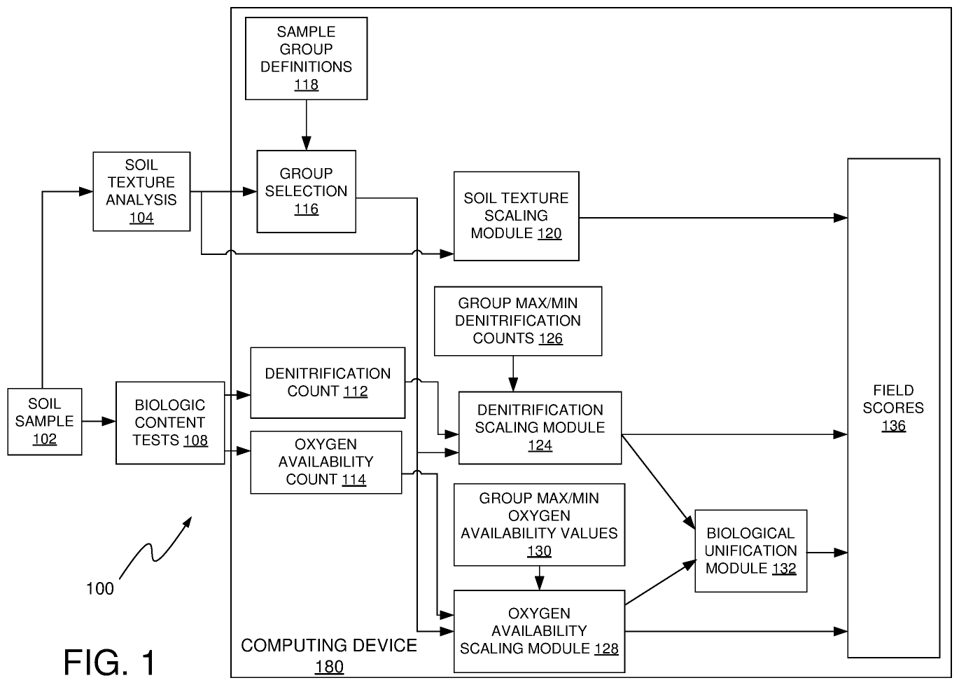

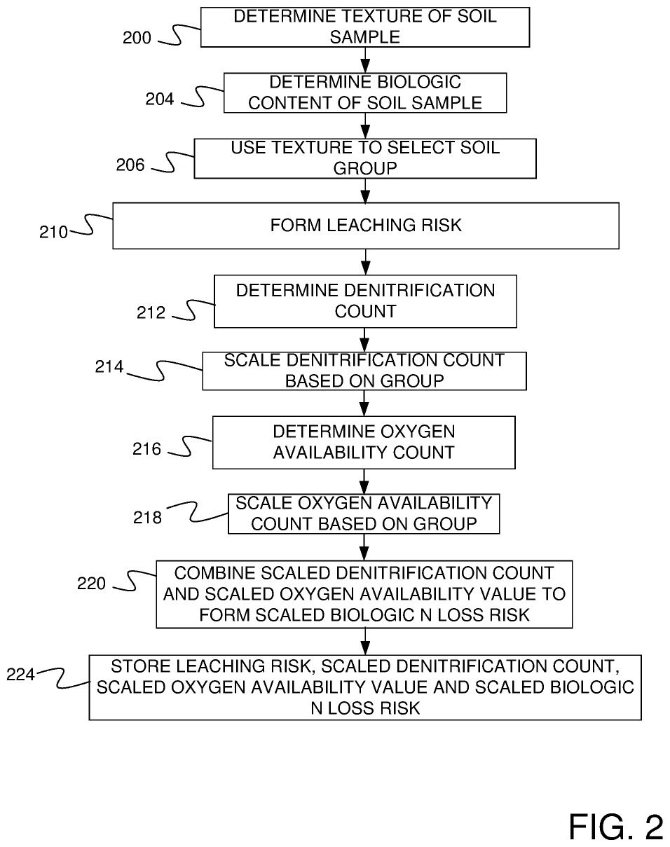

FIG. 1 provides a block diagram of a system 100 for generating nitrogen loss risk for a soil sample 102. FIG. 2 provides a method of processing a soil sample using system 100.

In step 200, an analysis 104 of soil sample 102 may be performed to determine the texture of the soil. In accordance with one embodiment, the texture of the soil is the percentages of clay, silt and sand in the soil and analysis 104 is performed based on the relative settling rates of clay, silt and sand in a liquid. Alternatively, the soil texture may come from other sources, including the USDA's SSURGO database. The SSURGO database contains information about soil as collected by a National Cooperative Soil Survey. The information can be displayed in tables or as maps and is available for most areas in the United States. Using the location where soil sample 102 was collected, the tables and maps can be used to obtain the texture for the soil sample.

In step 204, a portion of soil sample 102 is applied to biologic content tests 108 to determine the biologic content of the soil sample. In particular, biologic content tests 108 isolate genetic sequences of microbes in soil sample 102 so that various genetic sequences can be counted.

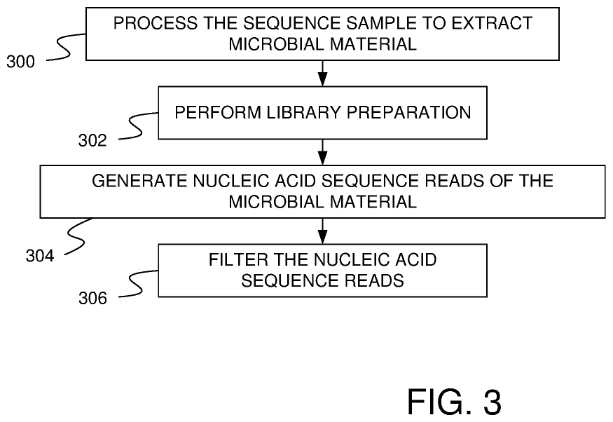

FIG. 3 provides a flow diagram of a method of performing biologic content tests 108. Soil sample 102 is processed at step 300 to extract microbial material (also referred to as microbial genetic material). In some embodiments, soil sample 102 may be stored at −80 degrees Celsius prior to extraction of the microbial material. In accordance with one embodiment, soil sample 102 is added to extraction vessels by mass, volume, suspension volume, or another measurement. Cell lysis is performed on the soil sample to release the microbial material including intracellular nucleic acids. Cell lysis may include chemical (buffers or salts), mechanical (bead beating or sonication), or thermal (e.g., freezing, free-thaw cycling, or microwaving) processes. Soil and the released microbial material are separated. Cellular debris may be removed using chemical precipitation or centrifugation. Additionally, contaminants may be removed using precipitation and elution of the microbial material. The microbial material may be prepared using fluorescent dyes or gels for downstream assay or spectroscopy.

In some embodiments, the nucleic acids of the microbial material may be processed prior to library preparation. For example, target genes or genome regions may be enriched for polymerase chain reaction (PCR) amplification or amplicon sequencing. Targeted DNA primers may be used to flank a region of interest. In some use cases, DNA fragment size may be controlled chemically using size selection gel beads, physically using ultrasonic shearing, or enzymatically using transposase fragmentation.

At step 302 sequencing library preparation is performed on the extracted microbial material. Library preparation may include attaching sequencing adapters or tags to nucleic acids to facilitate reading of the nucleic acids. Sequencing tags may be unique to each sample (e.g., serving as a barcode) and enable identification of sequenced data associated with each sample in a multiplexed run with multiple samples. Libraries may also be prepared using other suitable methods such as ligation or transposase. In some use cases, library preparation includes protocols from sequencer original equipment manufacturers (OEMs), third party kit providers, or other resources.

Once the sequencing library is prepared, the library or a portion of the library can be sequenced such that nucleic acid sequence reads of the microbial material are generated at step 304 using one or more techniques. In some embodiments, a sequencer performs sequencing (e.g., of DNA or RNA) and outputs sequence reads of the microbial material. In some embodiments, the nucleic acid sequence reads are generated using next generation sequencing (NGS) techniques including synthesis technology (ILLUMINA®), pyrosequencing (454 LIFE SCIENCES), ion semiconductor technology (Ion Torrent sequencing), single-molecule real-time sequencing (PACIFIC BIOSCIENCES®), or nanopore sequencing (OXFORD NANOPORE TECHNOLOGIES). DNA sequencing can also be performed as described in Sanger et al. (PNAS 74:5463, (1977)) and the Amersham International plc sequencing handbook, which methods are incorporated by reference herein.

At step 306, the nucleic acid sequence reads are filtered. For example, low quality sequence reads are discarded. Sequence reads can be considered of low quality by determining that a length of the sequence read is less than a threshold value, the sequence read includes at least a threshold number of ambiguous bases, or a read quality score (e.g., determined using a third-party tool) is less than a threshold score, for example.

Returning to FIG. 2, at step 206, the soil texture determined above is provided to a group selection module 116 executed by a processor (not shown) in a computing device 180. Group selection module 116 uses the soil texture and a set of sample group definitions 118 to select a sample group for soil sample 102. In accordance with one embodiment, each sample group is defined by a range of percentages for each of clay, silt and sand. In accordance with one embodiment, the sample groups are defined so that samples within each group have homogeneous characteristics with regard to Nitrogen loss.

At step 210, the soil texture is converted into a leaching risk having a value between zero and one hundred by a soil texture scaling module 120. In accordance with one embodiment, the leaching risk is calculated as fifty plus fifty times the difference between the percentage of sand and the percentage of clay. Thus, when there is an equal percentage of sand and clay, the leaching risk will be fifty. When the soil texture is 100% sand, the leaching risk will be one-hundred and when the soil texture is 100% clay, the leaching risk will be zero.

At step 212, a denitrification count module 112 executed by computing device 180 uses the biologic sequence reads produced by biologic content tests 108 at step 306 of FIG. 3 to determine a count of the genes available in the soil sample that contribute to denitrification. Lists of genes involved in denitrification are obtained and cross validated from multiple sources, including the MetaCyc database, the Kyoto Encyclopedia of Genes and Genomes (KEGG) and SEED gene ontologies. Further data sources are used to obtain additional gene annotation sources or models, including the UniProt, Pfam, and InterPro databases. These databases generally represent known molecular biology across organisms as organized for varying purposes which are not commonly organized to represent element cycling, soils, or agriculture.

As a general note, several of the most relevant genes are known to be horizontally transferred among microbes (such that organism names or name hierarchies are not necessarily deterministic of gene count). The number of these gene copies may vary within a given organism name or group (taxonomy).

In various embodiments, denitrification count module 112 assigns the sequence reads to the corresponding genes in the reference databases in order to determine counts of each gene. Denitrification count module 112 normalizes gene counts using total reads or gene hits, rarefaction, normalization by single copy marker genes, or other transformations. Denitrification count module 112 may combine reads or normalized read counts of subunits of a gene. For example, the counts of subunits of a gene are averaged in one embodiment to produce a count for the gene.

At step 214, the denitrification count produced by denitrification count module 112 is converted into a scaled denitrification score by a denitrification scaling module 124 executed by computing device 180. In one embodiment, this is performed by scaling the denitrification counts using a maximum denitrification count and a minimum denitrification count for the sample group of soil sample 102. Denitrification scaling module 124 retrieves the maximum and minimum denitrification counts for the sample group from group maximum/minimum denitrification counts 126. In accordance with one embodiment, the maximum and minimum denitrification counts are determined from denitrification counts of a collection of soil samples that fall within the sample group. In particular, the minimum denitrification count is calculated as the denitrification count of the soil sample marking the first quartile in the collection of soil samples for the group minus 1.5 times the interquartile difference in denitrification counts within the group. The maximum denitrification count is calculated as the denitrification count of the soil sample marking the third quartile in the collection of soil samples of the group plus 1.5 times the interquartile difference in denitrification counts within the group. The scaled denitrification count is then calculated as one hundred times the value of the denitrification count provided by denitrification count module 112 minus the minimum denitrification count for the group divided by the difference between the maximum denitrification count for the group minus the minimum denitrification count for the group. Thus, the scaled denitrification count has a value between zero and one hundred.

At step 216, oxygen availability count module 114 executed by computing device 180 uses the biologic sequence reads produced by biologic content tests 108 at step 306 of FIG. 3 to determine counts of genes in the soil sample that are associated with oxygen availability. In accordance with one embodiment, two gene types are counted. The first gene type is genes that are found in organisms that thrive in low-oxygen environments. Thus, a high count for these genes indicates low oxygen availability. The second gene type is genes that are found in organisms that require a large amount of oxygen in order to thrive. For such genes, higher gene counts mean more oxygen is available in the soil. Lists of genes of the first and second type are obtained and cross validated from multiple sources, including the MetaCyc database, the Kyoto Encyclopedia of Genes and Genomes (KEGG) and SEED gene ontologies. Further data sources are used to obtain additional gene annotation sources or models, including the UniProt, Pfam, and InterPro databases. These databases generally represent known molecular biology across organisms as organized for varying purposes which are not commonly organized to represent element cycling, soils, or agriculture.

As a general note, several of the most relevant genes are known to be horizontally transferred among microbes (such that organism names or name hierarchies are not necessarily deterministic of gene count). The number of these gene copies may vary within a given organism name or group (taxonomy).

In various embodiments, oxygen availability count module 114 assigns the sequence reads to the corresponding genes in the reference databases in order to determine counts of each gene. Oxygen availability count module 114 normalizes gene counts for each gene type using total reads or gene hits, rarefaction, normalization by single copy marker genes, or other transformations. Oxygen availability count module 114 may combine reads or normalized read counts of subunits of the genes of a gene type. For example, the counts of subunits of the genes a gene type are averaged in one embodiment to produce a count for the gene type.

Once the gene counts are determined, oxygen availability count module 114 combines the gene counts using a function to produce an oxygen availability value. In accordance with one embodiment, the count of genes of the first gene type is divided by the count of genes of the second gene type to produce the oxygen availability value.

At step 218, the oxygen availability value produced by oxygen availability count module 114 is converted into a scaled oxygen availability score by an oxygen availability scaling module 128 executed by computing device 180. In one embodiment, this is performed by scaling the oxygen availability value using a maximum oxygen availability value and a minimum oxygen availability value for the sample group of soil sample 102. Oxygen availability scaling module 128 retrieves the maximum and minimum oxygen availability values for the sample group from group maximum/minimum oxygen availability values 130. In accordance with one embodiment, the maximum and minimum oxygen availability values are determined from oxygen availability values of a collection of soil samples that fall within the sample group. In particular, the minimum oxygen availability value is calculated as the oxygen availability value of the soil sample marking the first quartile in the collection of soil samples for the group minus 1.5 times the interquartile difference in oxygen availability values within the group. The maximum oxygen availability value is calculated as the oxygen availability value of the soil sample marking the third quartile in the collection of soil samples of the group plus 1.5 times the interquartile difference in oxygen availability values within the group. The scaled oxygen availability value is then calculated as one hundred times the value of the oxygen availability value provided by oxygen availability count module 114 minus the minimum oxygen availability value for the group divided by the difference between the maximum oxygen availability value for the group minus the minimum oxygen availability value for the group. Thus, the scaled oxygen availability value has a value between zero and one hundred.

At step 220, a biologic unification module 132 executed by computing device 180 combines the scaled denitrification count with the scaled oxygen availability value to provide a scaled biologic N loss risk. In accordance with one embodiment, the scaled biologic N loss risk is formed as a weighted sum of the scaled denitrification count and the scaled oxygen availability value. In accordance with one embodiment, the weights used to form the scaled biologic N loss risk are selected so that the scaled biologic N loss risk has the same range of values as the leaching risk. For example, when the leaching risk has a range between zero and one hundred and the scaled denitrification count and the scaled oxygen availability value each have a range between zero and one hundred, the weights are selected such that the sum of the weights is one thereby making the scaled biologic N loss risk range between zero and one hundred. In one specific embodiment, the weights for the scaled denitrification count and the scaled oxygen availability value are the same.

At step 224, the leaching risk, the scaled denitrification count, the scaled oxygen availability value, and the scaled biologic N loss risk all stored in field scores 136 for the field that the soil sample was collected from. In accordance with some embodiments, each field is divided into multiple zones and the leaching risk, the scaled denitrification count, the scaled oxygen availability value, and the scaled biologic N loss risk are determined from a respective soil sample collected from the zone. The scores for each zone are stored in field scores 136 for the zones' field.

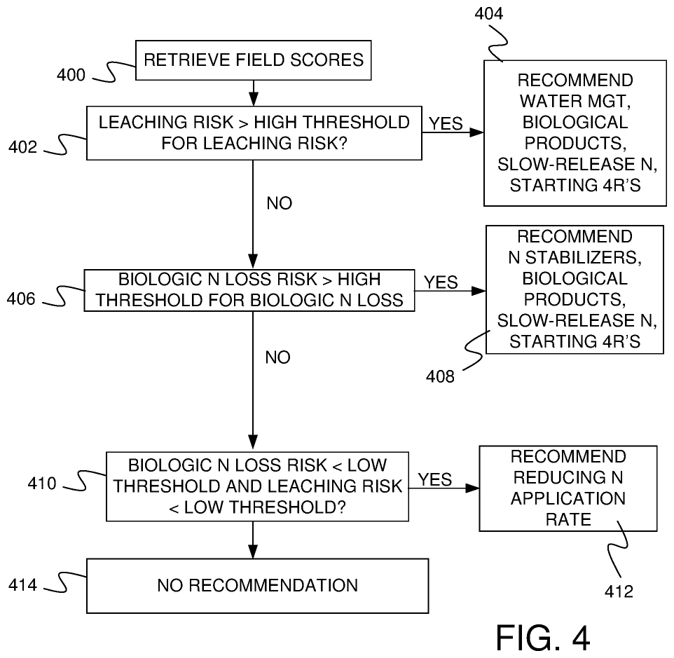

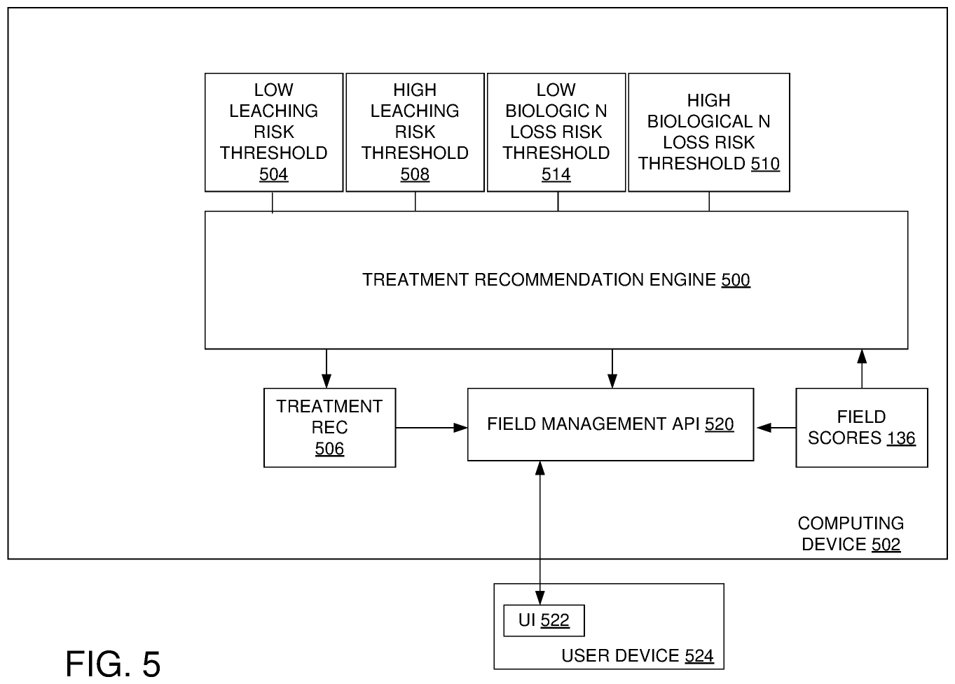

FIG. 4 provides a flow diagram of a method of identifying field treatments based on field scores 136. FIG. 5 provides a block diagram showing elements used in the method of FIG. 4. In the description below, the field treatments are discussed with reference to zones of a field. In embodiments where a field is not divided into zones, the entire field would be considered a single zone.

In step 400, a treatment recommendation engine 500 executed by a processor on a computing device 502 retrieves the field scores 136 for a zone. At step 402, treatment recommendation engine 500 compares the retrieved leaching risk for the zone to a high leaching risk threshold 508. If the leaching risk exceeds high leaching risk threshold 508, the zone has a sandy soil texture and there is a high risk of nitrogen loss through leaching. To address this risk, treatment recommendation engine stores a treatment recommendation 506 consisting of instituting water management, applying of one or more biological products, applying slow-release nitrogen and/or starting 4Rs management at step 404. Water management limits the amount of water applied to the zone to limit the amount of nitrogen that is transported out of the zone by excess water. The biologic products include biologic products that increase nitrogen availability in the rhizosphere, for example. Slow-release nitrogen is an application containing nitrogen in a form that slowly converts to nitrate. The “4R's” is a management technique that applies nitrogen using the Right fertilizer source, at the Right rate, at the Right time and at the Right place. In accordance with one embodiment, when the leaching risk exceeds high leaching risk threshold 508, treatment recommendation engine 500 does not recommend nitrogen stabilizers or nitrification inhibitors because these applications would also be leached form the soil due to high leaching risk.

If the leaching risk is below high leaching risk threshold 508, treatment recommendation engine 500 compares the scaled biologic N loss risk to a high biologic N loss risk threshold 510 at step 406. If the scaled biologic N loss risk exceeds high biologic N loss risk threshold 510, the zone is susceptible to N loss through biological functions. To address this risk, treatment recommendation engine stores a treatment recommendation 506 consisting of applying nitrogen stabilizers/nitrification inhibitors, applying one or more biological products, applying slow-release nitrogen and/or starting 4Rs management at step 408. Nitrogen stabilizers/nitrification inhibitors interfere with the conversion of ammonium to nitrate via nitrification. As a result, nitrate appears in the soil at a slower rate when such inhibitors are present. Since the rate at which microbes produce gaseous forms of nitrogen is limited by the rate at which nitrate appears in the soil, nitrogen stabilizers/nitrification inhibitors reduce the rate at which nitrogen is lost through biological functions.

If the scaled biologic N loss risk is less than high biologic N loss risk threshold 510 at step 406, treatment recommendation engine 500 determines whether the scaled biologic N loss risk is less than a low biologic N loss risk threshold 514 while the leaching risk is less than a low leaching risk threshold 504 at step 410. When both the scaled biologic N loss risk and the leaching risk are below these low thresholds, the total risk of nitrogen loss is low for the zone. As a result, the zone is unlikely to need as much nitrogen augmentation as other zones and treatment recommendation engine 500 therefore stores a treatment recommendation 506 consisting of reducing the amount of nitrogen applied to the zone at step 412.

If either the scaled biologic N loss risk is greater than low biologic N loss risk threshold 514 or the leaching risk is greater than low leaching risk threshold 504, the risk of nitrogen loss is acceptable and the zone is expected to have sufficient nitrogen. As a result, treatment recommendation engine 500 does not provide a treatment recommendation for the zone at step 414.

As discussed above, the scaled biologic N loss risk used in the method of FIG. 4 is formed from a scaled denitrification count and a scaled oxygen availability value. These scaled values were scaled based on denitrification counts and oxygen availability values of other soil samples assigned to the same sample group as the zone being processed in FIG. 4. Since the sample group is selected based on the soil texture of the zone, the scaling is based on the soil texture of the zone.

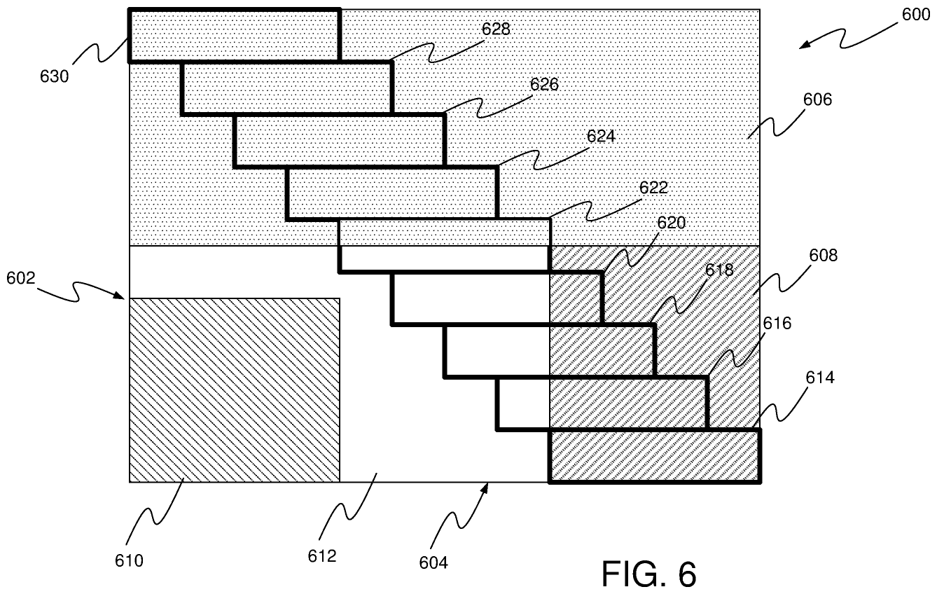

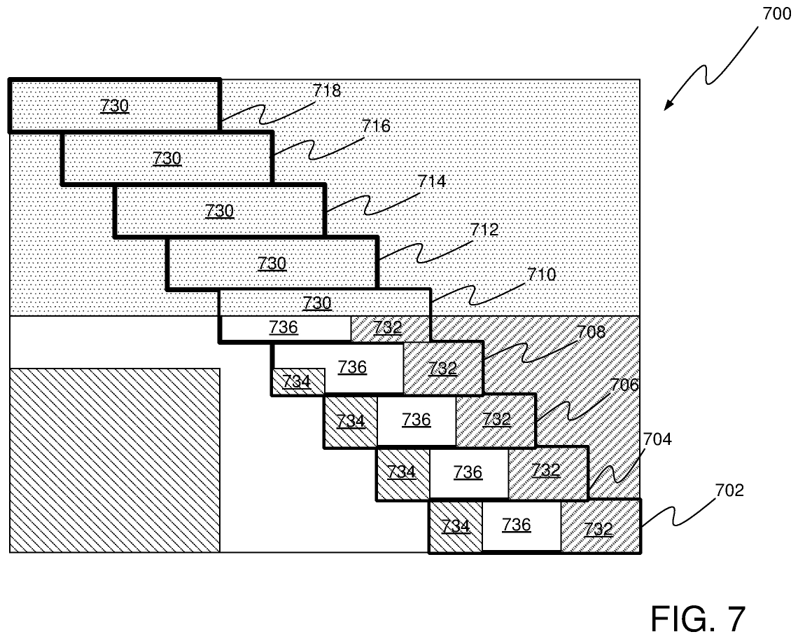

By using a scaled denitrification count and a scaled oxygen availability that are scaled based on the soil texture of the zone, the recommendations provided by treatment recommendation engine 500 are tailored for the soil texture of the zone. FIGS. 6 and 7 show the improvement in treatment recommendations that is achieved by scaling the denitrification count and the oxygen availability based on soil texture.

FIG. 6 provides a graph 600 with leaching risk shown along vertical axis 602 and an unscaled biologic N loss risk shown along horizontal axis 604. The unscaled biologic N loss risk is determined by forming the weighted sum of an unscaled denitrification count and an unscaled oxygen availability value. Thus, in FIG. 6, the scaling of the present embodiments has not been performed.

In graph 600, there are four treatment recommendation regions 606, 608, 610 and 612 that would result if unscaled biologic N loss risk was used in the method of FIG. 4 instead of the scaled biologic N loss risk. Treatment recommendation region 606 indicates combinations of leaching risk and unscaled biologic N loss risk that result in recommending instituting water management, the application of one or more biological products, the application of slow-release nitrogen and/or starting 4Rs management at step 404 of FIG. 4. Treatment recommendation region 608 indicates combinations of leaching risk and unscaled biologic N loss risk that result in recommending application of nitrogen stabilizers/nitrification inhibitors, the application of one or more biological products, the application of slow-release nitrogen and/or starting 4Rs management at step 408. Treatment recommendation region 610 indicates combinations of leaching risk and unscaled biologic N loss risk that result in recommending a reduction in the amount of nitrogen to be applied to the zone. Treatment recommendation region 612 indicates combinations of leaching risk and unscaled biologic N loss risk that results in no recommendation for the zone.

Applicants have discovered that the range of possible unscaled biologic N loss risk for a zone is correlated to the soil texture of the zone. Since the leaching risk is a direct function of the soil texture, there is a range of possible unscaled biologic N loss risk for each leaching risk.

In FIG. 6, treatment recommendation boxes 614, 616, 618, 620, 622, 624, 626, 628 and 630 show treatment recommendations that would be made for ranges of unsealed biologic N loss risk and leaching risk. For example, treatment recommendation box 614 shows that for all combinations of leaching risk and unscaled biologic N loss risk within treatment recommendation box 614, the treatment recommendations associated with region 608 will be recommended. Thus, within treatment recommendation box 614, the value of the unscaled biologic N loss risk for the zone is irrelevant to the treatment recommendation.

However, the amount of nitrogen to be applied to a zone is typically selected to overcome the various ways in which nitrogen is lost from that zone. As a result, ignoring the unscaled biologic N loss risk causes some zones to be treated with too much nitrogen and causes other zones to receive a biologic treatment when no such treatment is actually needed.

FIG. 7 provides a graph 700 showing treatment recommendation boxes 702, 704, 706, 708, 710, 712, 714, 716 and 718 for scaled biologic N loss risk overlayed on graph 600 of FIG. 6. Each treatment recommendation box is associated with a different sample group and provides treatment recommendations for combinations of leaching risk and scaled biologic N loss risk found in the sample group.

In FIG. 7, there are four possible treatment recommendation regions 730, 732, 734 and 736 that can appear in each treatment recommendation box based on the method of FIG. 4. Treatment recommendation region 730, which appears in treatment recommendation boxes 710, 712, 714, 716 and 718, indicates combinations of leaching risk and scaled biologic N loss risk that result in recommending instituting water management, the application of one or more biological products, the application of slow-release nitrogen and/or starting 4Rs management at step 404 of FIG. 4. Treatment recommendation region 732, which appears in treatment recommendation boxes 702, 704, 706, 708 and 710, indicates combinations of leaching risk and scaled biologic N loss risk that result in recommending application of nitrogen stabilizers/nitrification inhibitors, the application of one or more biological products, the application of slow-release nitrogen and/or starting 4Rs management at step 408. Treatment recommendation region 734, which appears in treatment recommendation boxes 702, 704, 706, and 708, indicates combinations of leaching risk and scaled biologic N loss risk that result in recommending a reduction in the amount of nitrogen to be applied to the zone. Treatment recommendation region 736, which appears in treatment recommendation boxes 702, 704, 706, 708 and 710, indicates combinations of leaching risk and scaled biologic N loss risk that results in no recommendation for the zone.

Comparing treatment recommendation box 702 to treatment recommendation box 614 shows some benefits of using a scaled biologic N loss risk instead of an unscaled biologic N loss risk. In particular, in treatment recommendation box 614 there is no recommendation region for reducing the amount of nitrogen applied to a zone. However, in treatment recommendation box 702 treatment recommendation region 734 is provided for reducing the amount of nitrogen applied to the zone. This allows the amount of nitrogen to be applied to a zone to be reduced when the amount of nitrogen planned for the zone would be too much for the risk of leaching and biologic N loss expected for the zone. Since using unscaled biologic N loss risk would not permit this type of recommendation, the embodiments that use a scaled biologic N loss risk result in improved recommendations for field treatment.

Similarly, in treatment recommendation box 614 there is no recommendation region for taking no action. However, in treatment recommendation box 702 treatment recommendation region 736 is provided for taking no action in the zone. This prevents the application of materials to a zone in which such applications would not significantly improve the availability of nitrogen to plants in the zone. Since using unscaled biologic N loss risk would not permit this type of recommendation, the embodiments that use a scaled biologic N loss risk result in improved recommendations for field treatment.

A field management API 520 is used to access measured characteristics of zones and treatment recommendations 506 stored for zones. Although field management API 520 is shown executing on computing device 502, in other embodiments, field management API 520 is executed on a separate server or on a user device.

In accordance with one embodiment, a user device 524 sends a request to field management API 520 for stored treatment recommendations for one or more zones. In response, field management API 520 retrieves the stored treatment recommendations, if any, and returns them to user device 524 so that the treatment recommendations can be displayed on user interface 522.

In accordance with another embodiment, upon a request from user device 524, field management API 520 retrieves one or more field maps that show fields associated with a current user of user device 524. Each field map includes a graphical depiction of one or more fields with lines representing the boundaries of each field and in some embodiments, additional lines depicting zones within each field. After retrieving a map, field management API 520 sets fill colors of the zones of each field to depict one or more measured characteristics of the zones.

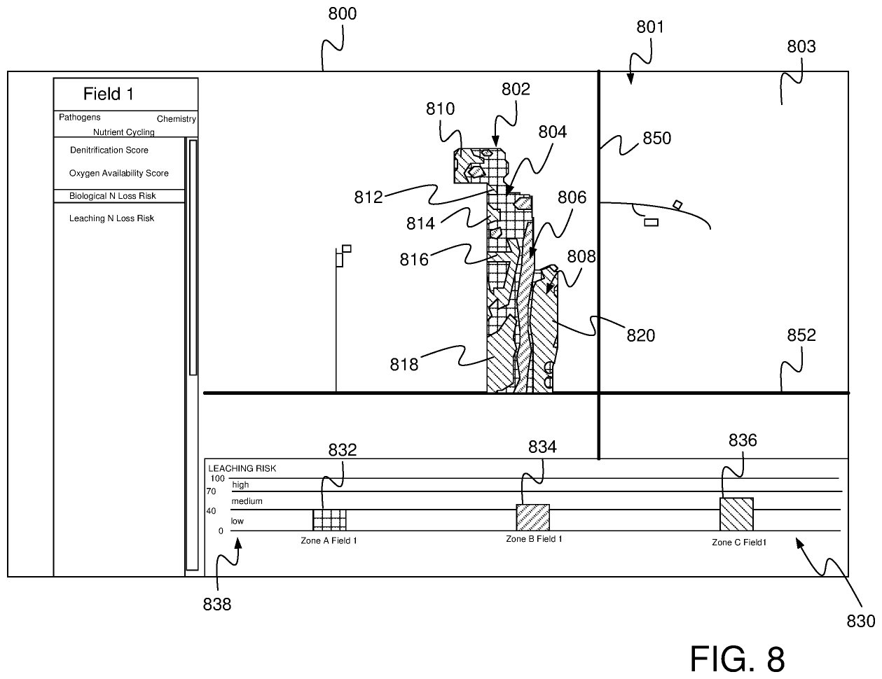

FIG. 8 provides an example user interface 800 showing a map 801 containing a field 802 showing scaled biologic N loss risks for zones in the field. Map 801 includes a satellite view 803 of a geographic area surrounding field 802 including, for example, roads 850 and 852. Field 802 is depicted as being divided into three zones 804, 806 and 808, which are each shown in a different color on user interface 800. (In FIG. 8, the different colors are represented using different hashing). An individual zone is not limited to being within a single closed zone boundary but instead can be constructed of multiple areas each defined by a separate respective zone boundary. For example, zone 808 covers noncontiguous areas 810, 812, 814, 816, 818 and 820. In accordance with one embodiment, each zone is formed of areas in the field that have similar characteristics.

User interface 800 also includes bar chart 830 that shows bars 832, 834 and 836 representing the respective scaled biologic N loss risks for zones 804, 806, and 808. Each bar has the same coloring as the corresponding zone shown in map 801. Bar chart 830 also includes designations 838 indicating whether the scaled biologic N loss risk for the zone is “high”, “medium”, or “low”.

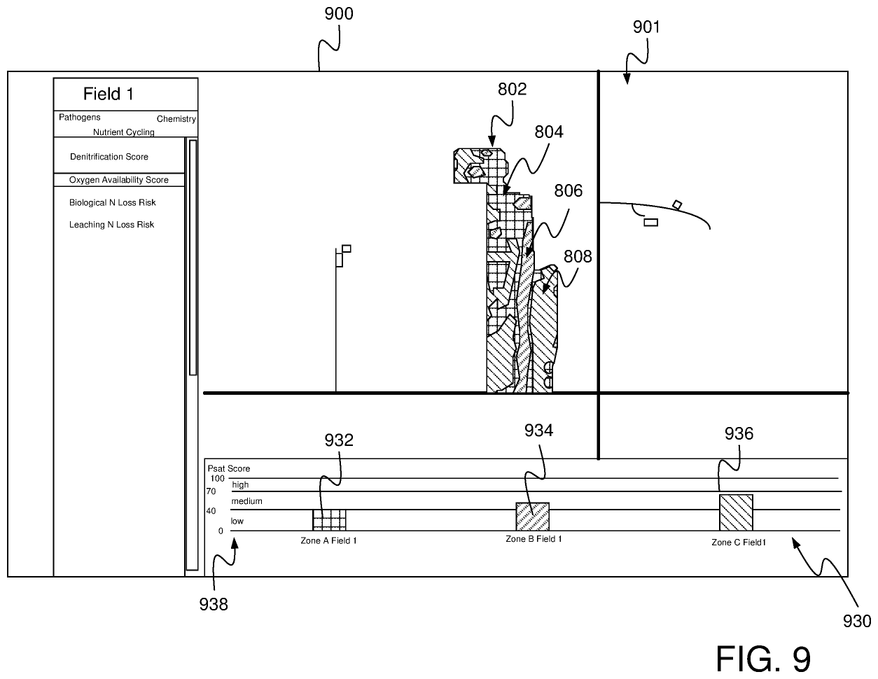

User interface 900 of FIG. 9 shows a map 901 that includes field 802 and depicts scaled oxygen availability for zones 804, 806 and 808. In FIG. 9, a bar chart 930 includes respective bars 932, 934 and 936 for zones 804, 806 and 808 depicting the scaled oxygen availability for each zone. Each bar has the same coloring as the corresponding zone shown in map 901. Bar chart 930 also includes designations 938 indicating whether the scaled oxygen availability for the zone is “high”, “medium”, or “low”.

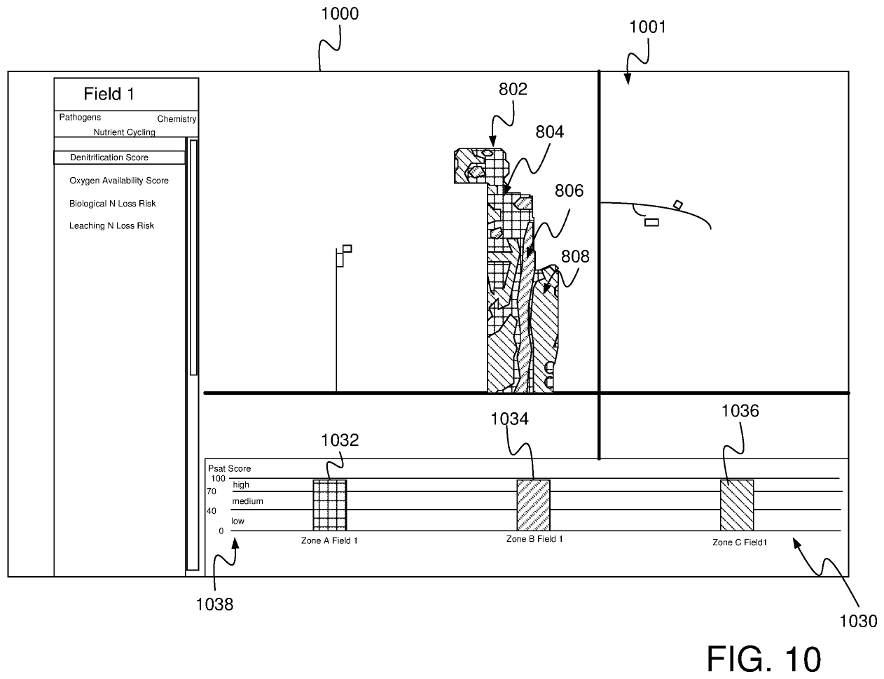

User interface 1000 of FIG. 10 shows a map 1001 that includes field 802 and depicts scaled denitrification counts for zones 804, 806 and 808. In FIG. 10, a bar chart 1030 includes respective bars 1032, 1034 and 1036 for zones 804, 806 and 808 depicting the scaled denitrification count for each zone. Each bar has the same coloring as the corresponding zone shown in map 1001. Bar chart 1030 also includes designations 1038 indicating whether the scaled denitrification count for the zone is “high”, “medium”, or “low”.

Comparing bar chart 830 to bar charts 930 and 1030, it can be seen that although the zones have a relatively high scaled denitrification count (bar chart 1030), indicating a large number of microbes that could perform denitrification, the scaled biologic N loss risk of the zones is relatively low (bar chart 830). This is due to a low oxygen availability score for the zones (bar chart 930) indicating that the zone is not a low-oxygen environment and as a result, the denitrification process is less likely to occur. Thus, the displayed scaled biologic N loss risks in FIG. 8 make it easier for farmers to quickly determine the true biologic N loss risk of different zones of field without requiring the farmer to separately determine the denitrification gene count and oxygen availability of each zone in the field.

The embodiments shown in FIGS. 8, 9 and 10 quickly convey to a farmer whether a treatment is required for a zone of a field. In particular, since the values depicted are scaled for groups of soil samples with common soil textures, they provide a better indication of the degree to which biologic N loss can be prevented in each zone and thus better convey to the farmer whether a treatment would improve the yield of the zone. For example, a high unscaled denitrification count may indicate to a farmer that a treatment should be applied to prevent denitrification. However, using the scaling technique described above, this unscaled value may be transferred into a scaled denitrification count that is low for soil samples with the same soil texture. In such a case, applying a treatment to lower the denitrification count would not be helpful since the denitrification count can only be lowered so far for a soil with this soil texture. By indicating directly that the denitrification count is low, the user interface of FIG. 10 quickly conveys to the farmer that no treatment is needed. Without the user interfaces of the present embodiments, farmers would have to guess as to whether the unscaled denitrification count warranted a treatment.

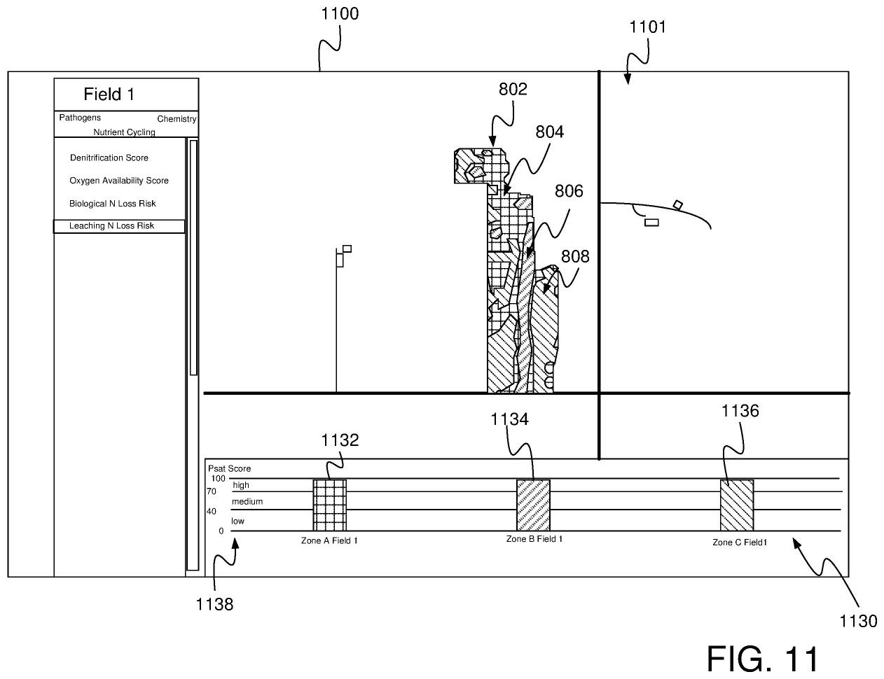

User interface 1100 of FIG. 11 shows a map 1101 that includes field 802 and depicts leaching risk for zones 804, 806 and 808. In FIG. 11, a bar chart 1130 includes respective bars 1132, 1134 and 1136 for zones 804, 806 and 808 depicting the nitrogen leaching risk for each zone. Each bar has the same coloring as the corresponding zone shown in map 1101. Bar chart 1130 also includes designations 1138 indicating whether the nitrogen leaching risk for the zone is “high”, “medium”, or “low”.

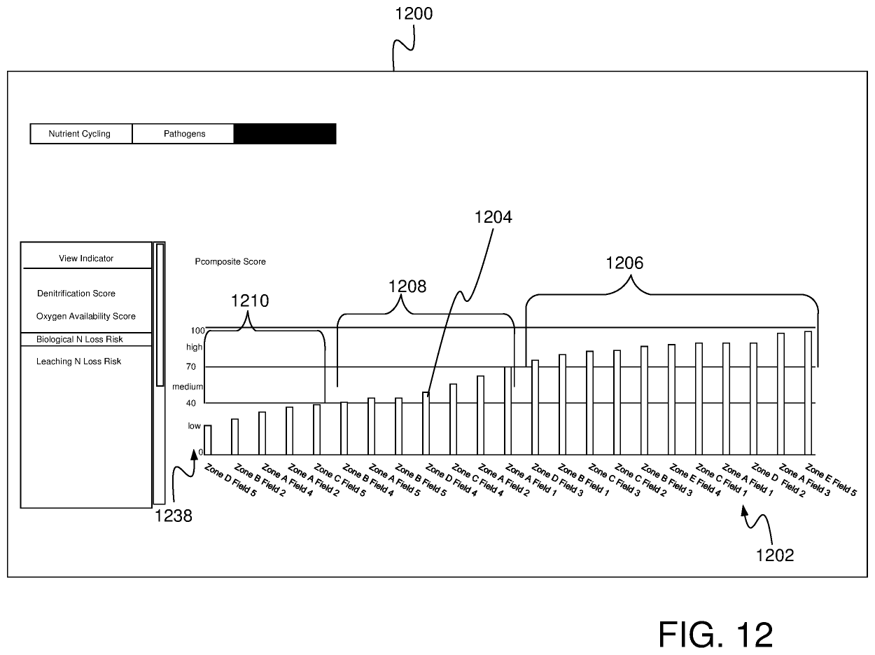

In other embodiments, the scaled biologic N loss risks for a collection of fields are shown together in a single user interface. FIG. 12 provides an example of such a user interface 1200, which provides a bar chart 1202 of scaled biologic N loss risk for each zone of a collection of fields. In user interface 1200, a separate bar is provided for each zone of the collection of fields. For example, bar 1204 is provided for zone D of Field 4. In addition, designations 1238 indicate whether the scaled biologic N loss risk for the zones are “high”, “medium”, or “low”. In FIG. 12, zones 1206 are shown to have scaled biologic N loss risks in the high range, zones 1208 are shown to have scaled biologic N loss risks in the medium range, and zones 1210 are shown to have scaled biologic N loss risks in the low range.

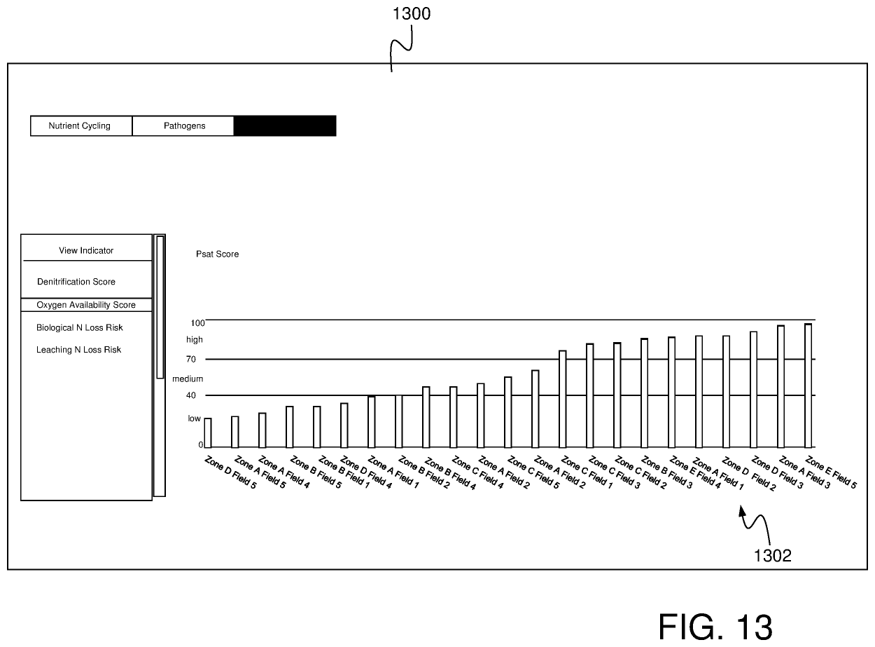

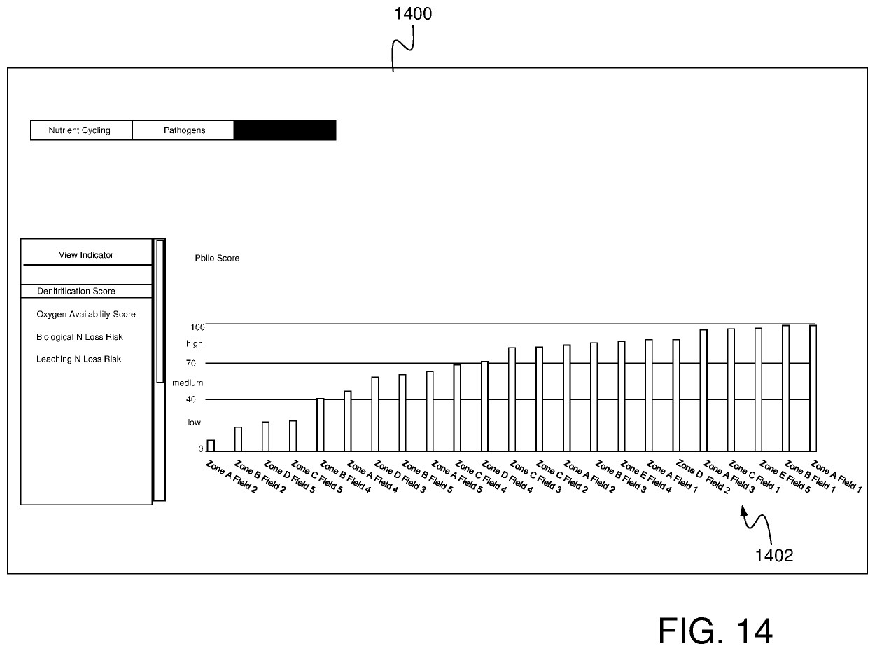

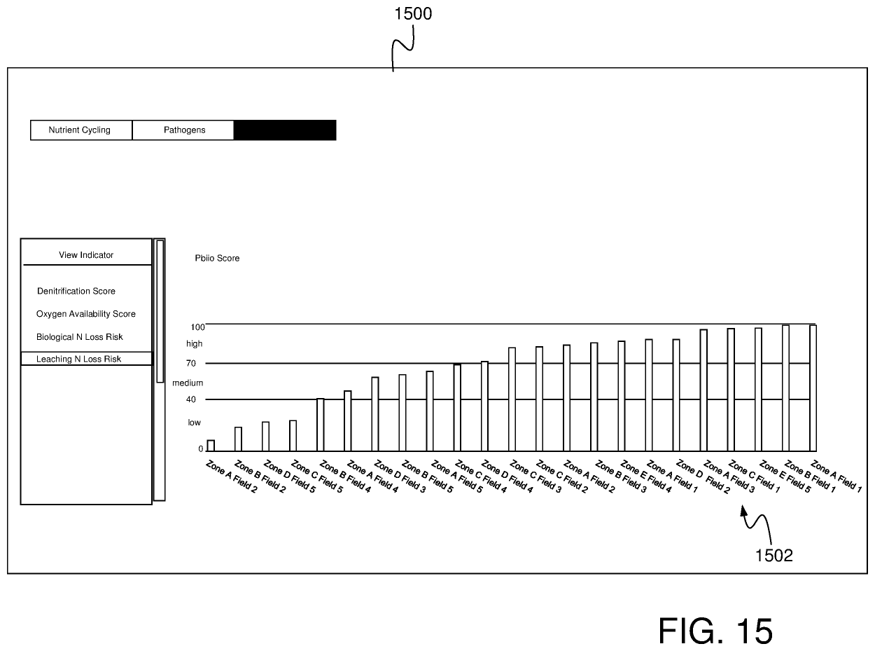

In further embodiments, field management API 520 also provides user interfaces 1300, 1400 and 1500 of FIGS. 13, 14, and 15, respectively, that provide bar charts 1302, 1402 and 1502 depicting scaled oxygen availability values, scaled denitrification counts and leaching risk, respectively, for the collection of zones shown in FIG. 12.

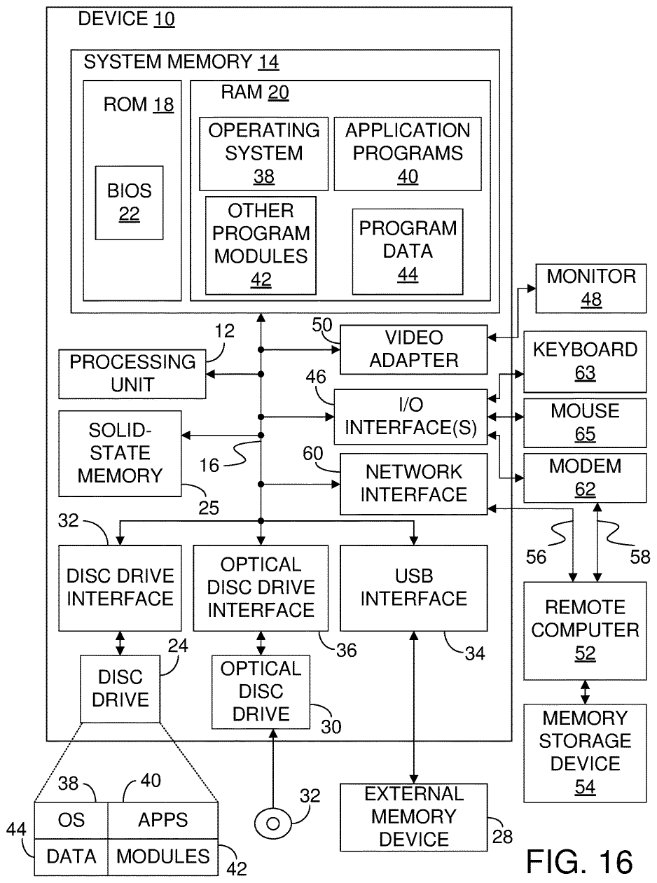

FIG. 16 provides an example of a computing device 10 that can be used to implement each of computing devices 180, 502 and 524 above. Computing device 10 includes a processing unit 12, a system memory 14 and a system bus 16 that couples the system memory 14 to the processing unit 12. System memory 14 includes read only memory (ROM) 18 and random-access memory (RAM) 20. A basic input/output system 22 (BIOS), containing the basic routines that help to transfer information between elements within the computing device 10, is stored in ROM 18. Computer-executable instructions that are to be executed by processing unit 12 may be stored in random access memory 20 before being executed.

Computing device 10 further includes an optional hard disc drive 24, an optional external memory device 28, and an optional optical disc drive 30. External memory device 28 can include an external disc drive or solid-state memory that may be attached to computing device 10 through an interface such as Universal Serial Bus interface 34, which is connected to system bus 16. Optical disc drive 30 can illustratively be utilized for reading data from (or writing data to) optical media, such as a CD-ROM disc 32. Hard disc drive 24 and optical disc drive 30 are connected to the system bus 16 by a hard disc drive interface 32 and an optical disc drive interface 36, respectively. The drives and external memory devices and their associated computer-readable media provide nonvolatile storage media for the computing device 10 on which computer-executable instructions and computer-readable data structures may be stored. Other types of media that are readable by a computer may also be used in the exemplary operation environment.

A number of program modules may be stored in the drives and RAM 20, including an operating system 38, one or more application programs 40, other program modules 42 and program data 44. In particular, application programs 40 can include programs for implementing the modules, engines and APIs discussed above. Program data 44 may include any data used by the systems and methods discussed above.

Processing unit 12, also referred to as a processor, executes programs in system memory 14 and solid-state memory 25 to perform the methods described above.

Input devices including a keyboard 63 and a mouse 65 are optionally connected to system bus 16 through an Input/Output interface 46 that is coupled to system bus 16. Monitor or display 48 is connected to the system bus 16 through a video adapter 50 and provides graphical images to users. Other peripheral output devices (e.g., speakers or printers) could also be included but have not been illustrated. In accordance with some embodiments, monitor 48 comprises a touch screen that both displays input and provides locations on the screen where the user is contacting the screen.

The computing device 10 may operate in a network environment utilizing connections to one or more remote computers, such as a remote computer 52. The remote computer 52 may be a server, a router, a peer device, or other common network node. Remote computer 52 may include many or all of the features and elements described in relation to computing device 10, although only a memory storage device 54 has been illustrated in FIG. 16. The network connections depicted in FIG. 16 include a local area network (LAN) 56 and a wide area network (WAN) 58. Such network environments are commonplace in the art.

The computing device 10 is connected to the LAN 56 through a network interface 60. The computing device 10 is also connected to WAN 58 and includes a modem 62 for establishing communications over the WAN 58. The modem 62, which may be internal or external, is connected to the system bus 16 via the I/O interface 46.

In a networked environment, program modules depicted relative to the computing device 10, or portions thereof, may be stored in the remote memory storage device 54. For example, application programs may be stored utilizing memory storage device 54. In addition, data associated with an application program may illustratively be stored within memory storage device 54. It will be appreciated that the network connections shown in FIG. 16 are exemplary and other means for establishing a communications link between the computers, such as a wireless interface communications link, may be used.

Although elements have been shown or described as separate embodiments above, portions of each embodiment may be combined with all or part of other embodiments described above.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms for implementing the claims.

What is claimed is:

1. A method of selecting a treatment recommendation for nitrogen loss in a field, the method comprising:
measuring a first characteristic of a soil sample that is related to biologic nitrogen loss to produce a measured value;
measuring a second characteristic of the soil sample;
using the second characteristic to select a sample group for the soil sample;
scaling the measured value based on the soil sample group the soil sample is placed into to form a scaled measure; and
using the scaled measure to select a treatment recommendation.

2. The method of claim 1 wherein the first characteristic comprises a count of genes involved in denitrification.

3. The method of claim 1 wherein the first characteristic comprises a value indicative of oxygen availability in the soil sample.

4. The method of claim 3 wherein the value indicative of oxygen availability comprises a count of genes associated with low oxygen availability.

5. The method of claim 3 wherein the value indicative of oxygen availability comprises a count of genes associated with high oxygen availability.

6. The method of claim 2 wherein the second characteristic comprises a soil texture of the soil sample.

7. The method of claim 6 wherein soil texture is derived from the USDA SSURGO database.

8. The method of claim 1 further comprising:
measuring a third characteristic of the soil sample, wherein the third characteristic is also related to biologic nitrogen loss to produce a second measured value;
scaling the second measured value based on the soil sample group the soil sample is placed into to form a second scaled measure; and
combining the second scaled measure with the scaled measure to form a combined measure;
wherein using the scaled measure to select the treatment recommendation comprises using the combined measure to select the treatment recommendation.

9. The method of claim 8 wherein the first characteristic comprises a count of genes involved in denitrification and the third characteristic comprises a value indicative of oxygen availability in the soil sample.

10. The method of claim 9 wherein the second characteristic comprises soil structure.

11. A method comprising:
using a soil texture of a soil sample to select a group of soil samples;
measuring a characteristic of the soil sample to produce a measured value;
scaling the measured value based on measured values determined for soil samples in the group of soil samples to form a scaled value;
displaying the scaled value instead of the measured value so as to improve a precision farming system.

12. The method of claim 11 wherein the characteristic comprises a denitrification count of the soil sample.

13. The method of claim 11 wherein the characteristic comprises an oxygen availability value.

14. The method of claim 11 wherein scaling the measured value comprises determining a maximum value for the characteristic for the group of soil samples, determining a minimum value for the characteristic for the group of soil samples and using the maximum and minimum to scale the measured value.

15. The method of claim 14 wherein determining the maximum value for the characteristic comprises determining a maximum value that is less than a largest value for the characteristic measured for a soil sample of the soil sample group.

16. A method comprising:
using a soil structure of a soil sample to select a sample group for the soil sample;
determining a denitrification count of the soil sample;
scaling the denitrification count relative to denitrification counts of other soil samples in the sample group to form a scaled denitrification count; and
displaying a user interface depicting the scaled denitrification count.

17. The method of claim 16 further comprising:
determining an oxygen availability value of the soil sample; and
scaling the oxygen availability value of the soil sample relative to oxygen availability values of other soil samples in the sample group to form a scaled oxygen availability value.

18. The method of claim 17 further comprising combining the scaled denitrification count and the scaled oxygen availability value to form a scaled biologic N loss risk.

19. The method of claim 18 further comprising determine a nitrogen leaching risk from the soil structure of the soil sample.

20. The method of claim 19 further comprising using the nitrogen leaching risk and the scaled biologic N loss risk to recommend a treatment for a zone of a field associated with the soil sample.
